(19) United States
(12) Patent Application Publication
(10) Pub. No.: US 2022/0346463 A1
(43) Pub. Date: Nov. 3, 2022

(54) CLINICAL GARMENT FOR INFANTS

(71) Applicant: Dominique M. Griffin, San Diego, CA (US)

(72) Inventor: Dominique M. Griffin, San Diego, CA (US)

(21) Appl. No.: 17/244,149

(22) Filed: Apr. 29, 2021

(51) Int. Cl. A41B 13/00

(52) U.S. Cl. CPC A41B 13/005

(57) ABSTRACT

An interactive one-piece pantsuit is provided for wear by an infant/toddler, when a medical device has been surgically implanted into the infant/toddler. For this purpose, the pantsuit acts as a protective garment which normally covers and protects extracorporeal components of the medical device. The garment, however, includes flaps which are positioned at key locations on the garment over the extracorporeal components of the medical device. These flaps can then be selectively raised to temporarily provide a caregiver with the necessary access to the medical device for operational or maintenance purposes.

### FIELD OF THE INVENTION

The present invention pertains generally to post-surgical garments for infants/toddlers. More particularly, the present invention pertains to garments to be worn by infants/toddlers that prevent the wearer from disconnecting or otherwise interfering with medical devices that have been connected to the wearer for medical purposes. The present invention is particularly, but not exclusively, useful as an interactive garment for infants/toddlers which provides a caregiver temporary access, through the garment, to key components of a medical device system for operational and maintenance purposes.

### BACKGROUND OF THE INVENTION

It happens all too often that children are sometimes born with serious anatomical difficulties that, for example, include tumors, deformities, and other functional disorders. Thanks to progress in the medical arts and sciences, however, many issues for the child (infant/toddler) can be surgically treated early on to give the child an excellent chance for an abundantly normal life. Of clinical importance, however, are cases where post-surgical care is necessary.

Typically, the person(s) primarily responsible for providing post-surgical rehabilitation for an infant/toddler is/are a parent(s), or other family member who may have little, if any, medical training. Nevertheless, even the most elementary post-surgical medical devices require a complete understanding of the steps needed to clean and maintain the particular device for its intended operational efficiency. Moreover, interference from the infant/toddler in these matters is to be avoided.

In addition to the knowledge that must be acquired to maintain a medical device, several other factors also contribute to the difficulties faced by a caregiver for a post-surgical infant/toddler. Of particular concern, insofar as the present invention is concerned, is a medical device (system) which contains an implanted component(s). Typically, such devices (systems) will also include extracorporeal components which must be routinely cleaned or replaced. The situation is further exacerbated by the fact the infant/toddler may be cold, tired, agitated, hungry, or think that he/she can somehow help the caregiver rearrange or disengage the extracorporeal components of the medical device.

For the reasons set forth above, it is an object of the present invention to provide a post-surgical garment for wear by an infant/toddler that prevents the wearer from disconnecting or otherwise interfering with a medical device that has been connected to him/her for medical purposes. Another purpose of the present invention is to provide a post-surgical garment for wear by an infant/toddler that gives a caregiver convenient and temporary access, through the garment, to key components of a medical device system for operational and maintenance purposes. Still another purpose of the present invention is to provide a post-surgical garment for wear by an infant/toddler that is easy to use, is simple to implement, and is comparatively cost effective.

### SUMMARY OF THE INVENTION

A pantsuit for wear by an infant/toddler following surgery that implants a medical device in the infant/toddler is designed to provide a caregiver direct access to components of the medical device that require periodic, post-surgical maintenance and/or replacement. With this in mind, the purpose of the pantsuit for the present invention is two-fold. For one, the comfort of the child during periodic maintenance of the medical device is a paramount concern. The other purpose is to facilitate the actions needed for a caregiver to perform the required maintenance procedures. In overview, the required procedures will typically include: periodic inspections of the medical device; required care and cleaning of the device; as well as using the medical device to infuse prescribed medications and/or withdraw blood samples from the infant/toddler for clinical evaluation.

Before describing the structural and functional aspects of the present invention, it is helpful to first appreciate and understand the medical device which necessitates the present invention. Of particular concern is the fact that a portion of the medical device is surgically implanted in the infant/toddler, and another portion of the medical device remains extracorporeal. Typically, the medical device will be a fluid tube that includes a dual lumen fluid line. Further, the fluid tube will have a distal end portion that is surgically implanted to establish fluid communication with the heart muscle of an infant/toddler. On the other hand, the proximal end of the fluid tube needs to remain extracorporeal for operational purposes.

The present invention appreciates that the time required, and the difficulties associated with the care and cleaning of a surgically implanted medical device, such as is envisioned for the present invention, will not be easily tolerated by an infant/toddler. Thus, an access garment in accordance with the present invention is provided that, for the most part, is comfortable for the infant/toddler. Importantly, however, the access garment also gives a caregiver access to the extracorporeal components of the medical device when required.

A key feature of the present invention is the inclusion of flaps that are appropriately located on the access garment. As intended for the present invention, when the access garment is being worn by an infant/toddler, these flaps are located to provide easy access to specific components of the medical device. Specifically, at a minimum, the access garment needs to include a dressing change flap and a device access flap. In detail, the dressing change flap is located on the access garment so it will be positioned over the incision site where the fluid tube exits from the body of the infant/toddler. On the other hand, the device access flap is located on the access garment so it will be positioned over components at the proximal end of the medical device which are needed for extracorporeal fluid communication with the heart muscle of the infant/toddler.

For an operation of the present invention, and infant/toddler is dressed in the access garment. When the infant/toddler is dressed, the flaps of the access garment are closed and secured by fasteners. It is only when care and cleaning of the incision site is required that the dressing change flap is unfastened and lifted to provide the caregiver access to the incision site. Similarly, it is only when medication is required for the infant/toddler, or it is necessary to withdraw blood from the infant/toddler, that the device access flap is unfastened and lifted to provide the caregiver syringe access to the medical device.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a front plan view of a post-surgical garment for wear by an infant/toddler in accordance with the present invention; and

FIG. 2 is a schematic drawing of an exemplary medical device for use with the present invention where, for purposes of clarity, subdermal portions of the device are shown as a dotted line and extracorporeal portions of the device are shown as a dashed line.

### DESCRIPTION OF THE PREFERRED EMBODIMENTS

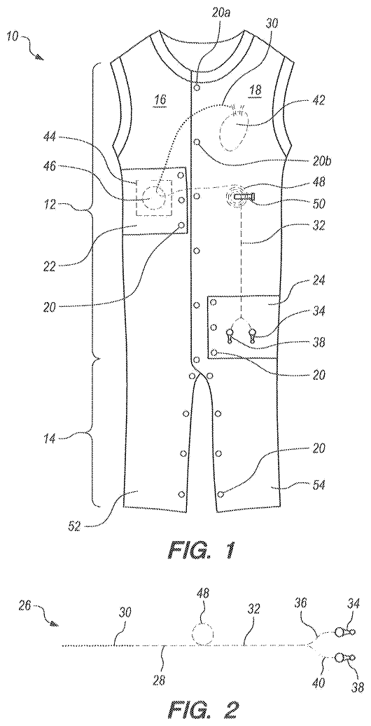

Referring initially to FIG. 1, an access garment for post-surgical wear by a child (infant/toddler) is shown and is generally designated 10. As shown, the access garment 10 includes a vest portion 12 that is intended to cover the upper body of the child (infant/toddler) (not shown), and it includes a pants portion 14 which is intended to cover the body of the infant/toddler below his/her waist. In this combination, the access garment 10 is essentially a one-piece pantsuit.

Still referring to FIG. 1, the vest portion 12 of the access garment 10 is shown to include an under-panel 16 and an over-panel 18. Specifically, in use, the under-panel 16 is positioned to cover the torso of the child (infant/toddler). Once the under-panel 16 is in place, the over-panel 18 is folded over the under-panel 16 and fasteners 20a and 20b (et. al.) are connected to secure the vest portion 12 of the access garment 10 on the child (infant/toddler). At this point it is to be noted that (as shown in FIG. 1) the present invention envisions numerous fasteners 20, being used at as many different locations on the access garment, to collectively secure the access garment 10 on the child (infant/toddler). Moreover, it is also to be noted that the fasteners 20 can be of any type well known in the pertinent art, such as clamps, buttons, snaps, ties, and straps.

FIG. 1 also shows that the access garment 10 includes a dressing change flap 22 and a device access flap 24. As used for the present invention, these flaps 22 and 24 are specifically located to provide easy access to areas on the body of the child (infant/toddler) while s/he is wearing the access garment 10. Although the flaps 22 and 24 are shown at typical locations in FIG. 1, it is to be appreciated that an access garment 10 can be provided which positions the flaps 22 and 24 in different locations. It is also envisioned that there may be additional flaps (not shown), at different locations on the access garment 10, depending on the particular needs of the child (infant/toddler).

Separate from the access garment 10, FIG. 2 shows a typical post-surgical medical device, generally designated 26, portions of which may be implanted in an infant/toddler during surgery. As shown, the typical medical device 26 is a dual lumen, fluid tube 28 which has a subcutaneous section 30 at the distal end of the fluid tube 28 (shown as a dotted line), and an extracorporeal section 32 at the proximal end of the fluid tube 28 (shown as a dashed line). FIG. 2 also shows that a first connector 34 (i.e., hub) is connected for fluid communication with the first lumen 36 of the fluid tube 28, and that a second connector 38 (i.e., hub) is connected for fluid communication with the second lumen 40 of the fluid tube 28.

Returning to FIG. 1 an installation of the medical device 26 onto the infant/toddler is shown relative to the access garment 10 for disclosure purposes. There it will be seen that the distal end of the subcutaneous section 30 of the fluid tube 28 is connected in fluid communication with the heart muscle 42 (shown in phantom) of the infant/toddler. The subcutaneous section 30 of the fluid tube 28 is then positioned between the heart muscle 42 and a predetermined incision site 44. At the incision site 44, the subcutaneous section 30 of the fluid tube 28 emerges from the chest of the infant/toddler to continue therefrom as the extracorporeal section 32 of the fluid tube 28. A bio-patch protective disk 46 is adhesively positioned against the body of the infant/toddler to protect the incision site 44 and to anchor the fluid tube 28 as it exits the chest of the infant/toddler.

Still referring to FIG. 1, if the extracorporeal section 32 of the fluid tube 28 is determined to be too long, part of the extracorporeal section 32 may be formed as a coil 48 which can be held stationary by a security strap 50 located on the inside of the over-panel 18. Specifically, the coil 48 can be created to adjust the location of the connectors 34 and 38 for their proper placement under the device access flap 24.

A method for maintaining a medical device 26 which has been surgically implanted in an infant/toddler, includes covering the upper body of the infant/toddler with the access garment 10. In detail, this requires engaging the under-panel 16 with the overlapping over-panel 18 using fasteners 20 to establish a secure engagement wherein the vest portion 12 wraps completely around the upper body of the infant/toddler. As envisioned for the present invention, the access garment 10 can be worn continuously, if desired.

Maintaining the medical device 26 involves lifting the dressing change flap 22 to establish access to the incision site 44 on the chest of the child. Once the dressing change flap 22 has been lifted the user can proceed to clean and dress the incision site 44. This will typically include the need to replace the protective disk 46 at the incision site 44. In a separate operation, the device access flap 24 can be lifted to establish access to the first connector 34 and the second connector 38 at the proximal end of the medical device 26.

As indicated above, the medical device 26 will typically include a dual lumen fluid tube 28 with each lumen 36, 40 having a proximal end and a distal end. The distal subcutaneous section 30 of the fluid tube 28 is surgically connected in fluid communication with the heart muscle 42 of the child, and the fluid tube 28 extends subcutaneously from the heart muscle 42 to the surgical incision site 44. In accordance with the present invention, it is envisioned that a protective surgical dressing (e.g., a protective disk 46) is secured to the child over the surgical incision site 44 by an adhesive. Lifting the dressing change flap 22 at the incision site 44 allows the user to change the protective surgical dressing (i.e., protective disk 46) and the adhesive that secure the fluid tube 28 at the surgical incision site 44. Once the area is cleaned and the emplacement of a new protective surgical dressing (protective disk) 46 has been completed, the user simply secures the appropriate fasteners 20.

As envisioned for the present invention, the fluid tube 28 is a dual lumen line, with both lumens 36, 40 respectively extending between the distal end and the proximal end of the fluid tube 28. Similar to the operation of the dressing change flap 22 disclosed above, lifting the device access flap 24 provides the user access to a component(s) of the medical device 26 for maintenance and/or operational purposes. In the case of the device access flap 24, the components of interest are the fluid connectors 34 and 38. Importantly, by design each lumen 36, 40 of the fluid tube 28 has a specific purpose. One lumen is used to infuse a fluid medicament to the infant/toddler, and the other is used to withdraw blood from the heart muscle 42 of the infant/toddler.

As shown in FIGS. 1 and 2, each fluid connector 34 and 38 is affixed for fluid communication with a respective lumen 36 and 40 of the fluid tube 28. In use, separate syringes (not shown) are respectively engaged with a connector for a specific purpose i.e., infusing a medicament or withdrawing blood from the heart muscle 42 of the infant/toddler. For convenience, the connectors are typically color-coded with a yellow connector indicating a use for infusing medications and a blue connector indicating a use for withdrawing blood. In each case, the respective lumens need to be flushed with a saline solution immediately after use.

In a preferred embodiment of the access garment 10, a pants portion 14 is included. As shown in FIG. 1, the pants portion 14 of the access garment 10 extends below the vest portion 12 with a first pantleg 52 and a second pantleg 54 for individually surrounding a respective leg of the child to hold the pantsuit securely on the child.

While the particular Clinical Garment for Infants as herein shown and disclosed in detail is fully capable of obtaining the objects and providing the 5 advantages herein before stated, it is to be understood that it is merely illustrative of the presently preferred embodiments of the invention and that no limitations are intended to the details of construction or design herein shown other than as described in the appended claims.

What is claimed is:

1. An access garment for a young child which comprises:
a one-piece pantsuit including a vest portion and a pants portion, wherein the vest portion wraps around the upper body of the child, with the vest portion having an under-panel covering a portion of the child's chest and an overlapping over-panel with fasteners for establishing a secure engagement between the under-panel and the over-panel on the chest of the child, and wherein the pants portion of the pantsuit extends below the vest portion with a first part and a second part for individually surrounding a respective leg of the child to hold the pantsuit securely on the child;
a dressing change flap created on a selected panel of the vest portion and moveable thereon for a secure engagement therewith, wherein the dressing change flap is positioned to cover a surgical incision on the chest of the child, and wherein the dressing change flap can be disengaged from the vest portion and lifted to provide access to the surgical incision to clean and dress the incision; and
an access flap created on a selected panel of the vest portion and moveable thereon for a secure engagement therewith, wherein the access flap is positioned to cover preselected components of a medical device, and wherein the access flap can be disengaged from the vest portion and lifted to provide access to the medical device components for operational purposes associated with the medical device.

2. The access garment of claim 1 wherein the medical device comprises:
a fluid tube having at least one lumen with a proximal end and a distal end, wherein the distal end is surgically connected in fluid communication with the heart muscle of the child and the fluid tube extends subcutaneously from the heart muscle to the surgical incision and extracorporeally from the surgical incision to the proximal end of the fluid tube;
a protective surgical dressing positioned over the surgical incision and a portion of the fluid tube emerging from the surgical incision, wherein the surgical dressing and the emerging portion of the fluid tube are secured against the chest of the child by an adhesive member; and
a fluid connector attached to the proximal end of the fluid tube in fluid communication with the lumen of the fluid tube to establish a fluid pathway from a medical syringe, through the lumen of the fluid tube, to the heart muscle.

3. The access garment of claim 2 wherein the fluid tube is a dual lumen line having a first lumen and a second lumen respectively extending between the distal end and the proximal end of the fluid tube.

4. The access garment of claim 3 wherein the first lumen is for infusing a fluid medicament to the child and the second lumen is for withdrawing blood from the heart muscle of the child.

5. The access garment of claim 4 wherein the first lumen and the second lumen are flushed with a saline solution immediately after use.

6. The access garment of claim 5 wherein the fluid connector includes a pair of hubs with a first hub affixed for fluid communication with the first lumen of the fluid tube and a second hub affixed for fluid communication with the second lumen of the fluid tube.

7. The access garment of claim 6 wherein the pair of hubs are color-coded with a yellow hub indicating a use for infusing medications and a blue hub indicating a use for withdrawing blood.

8. The access garment of claim 2 further comprising a security strap affixed to the inside of the over-panel and positioned between the over-panel and the under-panel to hold and secure a coil of the fluid tube between the surgical incision and the access flap.

9. A method for maintaining a medical device surgically implanted in an infant/toddler, which comprises the steps of:
outfitting the infant/toddler with a one-piece pantsuit having a vest portion, wherein the vest portion has an under-panel covering a portion of the infant's/toddler's chest, and an overlapping over-panel with fasteners for establishing a secure engagement between the under-panel and the over-panel when the vest portion wraps around the upper body of the infant/toddler to cover the chest of the infant/toddler;
lifting a dressing change flap created on a selected panel of the vest portion, wherein the dressing change flap is positioned to cover a surgical incision on the chest of the infant/toddler, and wherein the dressing change flap is lifted to provide access to the surgical incision to clean and dress the incision; and
lifting an access flap created on a selected panel of the vest portion, wherein the access flap is positioned to cover preselected components of the medical device, and wherein the access flap is lifted to provide access to the medical device components for operational purposes associated with the medical device.

10. The method of claim 9 wherein the medical device comprises:
a fluid tube having at least one lumen with a proximal end and a distal end, wherein the distal end is surgically connected in fluid communication with the heart muscle of the infant/toddler and the fluid tube extends subcutaneously from the heart muscle to the surgical incision and extracorporeally from the surgical incision to the proximal end of the fluid tube;
a protective surgical dressing positioned over the surgical incision and a portion of the fluid tube emerging from the surgical incision, wherein the surgical dressing and the emerging portion of the fluid tube are secured against the chest of the infant/toddler by an adhesive member; and
a fluid connector attached to the proximal end of the fluid tube in fluid communication with the lumen of the fluid tube to establish a fluid pathway from a medical syringe, through the lumen of the fluid tube, to the heart muscle.

11. The method of claim 10 wherein the step of lifting the dressing change flap further comprises the step of changing the protective surgical dressing which is used to secure the portion of the fluid tube emerging from the surgical incision against the chest of the infant/toddler.

12. The method of claim 10 wherein the fluid tube is a dual lumen line having a first lumen and a second lumen respectively extending between the distal end and the proximal end of the fluid tube and wherein the step of lifting the access flap provides access to the fluid connector to perform the steps of:
infusing a fluid medicament through the first lumen to the infant/toddler; and
withdrawing blood from the heart muscle of the infant/toddler through the second lumen.

13. The method of claim 12 wherein the first lumen and the second lumen are flushed with a saline solution immediately after use.

14. The method of claim 13 wherein the fluid connector includes a pair of hubs with a first hub affixed for fluid communication with the first lumen of the fluid tube and a second hub affixed for fluid communication with the second lumen of the fluid tube, and wherein the pair of hubs are color-coded with a yellow hub indicating a use for infusing medications and a blue hub indicating a use for withdrawing blood.

15. The method of claim 14 wherein the pants portion of the pantsuit extends below the vest portion with a first pantleg and a second pantleg for individually surrounding a respective leg of the infant/toddler to hold the pantsuit securely on the infant/toddler.

16. A method for manufacturing an access garment, to facilitate operating and maintaining an implanted medical device during a post-surgical rehabilitation of an infant/toddler, which comprises the steps of:
fabricating a one-piece pantsuit for wear by an infant/toddler, wherein the pantsuit has a vest portion with an under-panel covering a portion of the infant's/toddler's chest and an overlapping over-panel with fasteners for establishing a secure engagement between the under-panel and the over-panel to wrap the vest portion around the upper body of the infant/toddler;
creating a dressing change flap on a selected panel of the vest portion, wherein the dressing change flap is positioned on the pantsuit to cover a surgical incision on the chest of the infant/toddler;
providing fasteners on the dressing change flap to hold the dressing change flap against the vest portion, wherein the fasteners can be disengaged from the vest portion to lift the dressing change flap for access to the surgical incision to clean and dress the incision;
creating an access flap on a selected panel of the vest portion, wherein the access flap is positioned on the pantsuit to cover preselected extracorporeal components of a medical device; and
providing fasteners on the access flap to hold the access flap against the vest portion, wherein the fasteners can be disengaged from the vest portion to lift the access flap for access to the medical device components for operational purposes associated with the medical device.

17. The method of claim 16 further comprising the step of incorporating a pants portion of the pantsuit wherein the pants portion extends below the vest portion with a first pantleg and a second pantleg for individually surrounding a respective leg of the infant/toddler to hold the pantsuit securely on the infant/toddler.

18. The method of claim 16 further comprising the step of incorporating a security strap affixed to the inside of the over-panel and positioned between the over-panel and the under-panel to hold and secure a coil of the fluid tube between the surgical incision and the access flap.

19. The method of claim 16 wherein the pantsuit is made of a textile material.

20. The method of claim 16 wherein the fasteners for the pantsuit are selected from the group consisting of clamps, buttons, snaps, ties, and straps.
